(19) United States
(12) Patent Application Publication
(10) Pub. No.: US 2024/0342401 A1
(43) Pub. Date: Oct. 17, 2024

(54) APPARATUS AND METHODS FOR GAS VERIFICATION

(71) Applicant: Alcon Inc., Fribourg (CH)

(72) Inventor: Conrad SAWICZ, Tustin, CA (US)

(21) Appl. No.: 18/750,857

(22) Filed: Jun. 21, 2024

Related U.S. Application Data
(62) Division of application No. 17/491,919, filed on Oct. 1, 2021.
(60) Provisional application No. 63/090,887, filed on Oct. 13, 2020.

(51) Int. Cl. A61M 13/00; A61F 9/007

(52) U.S. Cl. CPC A61M 13/00; A61F 9/007; A61M 2202/02; A61M 2205/3368; A61M 2210/0612

(57) ABSTRACT

Embodiments described herein generally relate to apparatus and methods for verifying the concentration of mixed gas to be used for ophthalmic administration. In an embodiment is provided a method that includes introducing a first portion of mixed gas into a chamber, determining a temperature change of the first portion which is indicative of a parameter of the first portion, determining that the parameter satisfies a predetermined value, and introducing a second portion of mixed gas into a patient's eye. In another embodiment is provided a method that includes exposing an element inside a chamber to a first portion of mixed gas, determining a change in a physical characteristic of the element that changes in response to a stimulus, the physical characteristic indicative of a parameter of the first portion, determining that the parameter satisfies a predetermined value, and introducing a second portion of mixed gas into a patient's eye.

### PRIORITY CLAIM

This application is a divisional of U.S. patent application Ser. No. 17/491,919, titled “APPARATUS AND METHODS FOR GAS VERIFICATION,” filed Oct. 1, 2021, whose inventor is Conrad Sawicz, which claims the benefit of priority of U.S. Provisional Patent Application Ser. No. 63/090,887 titled “APPARATUS AND METHODS FOR GAS VERIFICATION,” filed on Oct. 13, 2020, whose inventor is Conrad Sawicz, which is hereby incorporated by reference in its entirety as though fully and completely set forth herein.

### BACKGROUND

### Field

Embodiments of the present disclosure generally relate to apparatus and methods for verifying the concentration of a mixed gas to be used for ophthalmic administration.

### Description of the Related Art

Methods for repairing, e.g., a retinal detachment, typically involve the use of one or more gases. One method for retinal detachment repair is pneumatic retinopexy. Pneumatic retinopexy typically involves intravitreal injection of an expanding, long-acting intraocular gas such as sulfur hexafluoride (SF₆), carbon tetrafluoride (CF₄), hexafluoroethane (C₂F₆), or octafluoropropane (C₃F₈) diluted in air, after which laser treatment or cryopexy is applied to the retinal tear. Briefly, the patient's head is positioned such that the expanding gas bubble presses against the detached retina and pushes it back into place. The surface tension of the water/gas interface helps prevent fluid movement into the retinal breaks and to seal the retina in place, allowing for chorioretinal adhesion. One technique for treating retinal detachment includes pars plana vitrectomy (PPV). Briefly, PPV involves removing of the vitreous gel and vitreoretinal traction, locating and lasing retinal tears, and inserting an intraocular gas tamponade, e.g., 20% SF₆ in air or 14% C₃F₈ in air.

Typically, the intraocular gas is drawn into a syringe, diluted with air, and mixed manually prior to the retinal detachment repair procedure. The volumes of gases to achieve the appropriate concentrations (or dilution ratios) are calculated based on varying syringe sizes. Achieving appropriate concentrations, however, remains a challenge.

There is a need for apparatus and methods for verifying the concentration of a mixed gas to be used for ophthalmic administration.

### SUMMARY

Embodiments of the present disclosure generally relate to apparatus and methods for verifying the concentration of a mixed gas to be used for ophthalmic administration.

In an embodiment is provided a method that includes preparing a volume of mixed gas, the volume of mixed gas comprising a first portion and a second portion, introducing the first portion into a chamber, determining a temperature change or a rate of temperature change of the first portion, the temperature change or the rate of temperature change indicative of a parameter of the first portion, determining that the parameter satisfies a predetermined value, and introducing the second portion into a a patient's eye (e.g., the vitreous chamber of the patient's eye).

In another embodiment is provided a method that includes preparing a volume of mixed gas, the volume of mixed gas comprising a first portion and a second portion, introducing the first portion into a first chamber, introducing a reference sample into a second chamber, and exposing the first portion and the reference sample to the same conditions. The method further includes determining a first rate of temperature change of the first portion, determining a second rate of temperature change of the reference sample, measuring a difference between the first rate of temperature change and the second rate of temperature change, the difference between the first rate of temperature change and the second rate of temperature change indicative of a parameter of the first portion, determining that the parameter satisfies a predetermined value, and introducing the second portion into a patient's eye (e.g., the vitreous chamber of the patient's eye).

In another embodiment is provided a method that includes preparing a volume of mixed gas, the volume of mixed gas comprising a first portion and a second portion, introducing the first portion into a chamber, the chamber housing an element, determining a change in a physical characteristic of the element that changes in response to temperature or electron excitation, the physical characteristic indicative of a parameter of the first portion, determining that the parameter satisfies a predetermined value, and introducing the second portion into a patient's eye (e.g., the vitreous chamber of the patient's eye).

### BRIEF DESCRIPTION OF THE DRAWINGS

So that the manner in which the above-recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 is an example apparatus for verifying a parameter of a mixed gas according to at least one embodiment of the present disclosure.

FIG. 2 is a flowchart of an example method of verifying a parameter of a mixed gas according to at least one embodiment of the present disclosure.

FIG. 3 is an example apparatus for verifying a parameter of a mixed gas according to at least one embodiment of the present disclosure.

FIG. 4 is a flowchart of an example method of verifying a parameter of a mixed gas according to at least one embodiment of the present disclosure.

FIG. 5 is an example apparatus for verifying a parameter of a mixed gas according to at least one embodiment of the present disclosure.

FIG. 6 is a flowchart of an example method of verifying a parameter of a mixed gas according to at least one embodiment of the present disclosure.

### DETAILED DESCRIPTION

Embodiments of the present disclosure generally relate to apparatus and methods for verifying the concentration of a mixed gas to be used for ophthalmic administration. The apparatus and methods described herein can be used with ophthalmic procedures such as retinal detachment repair.

Typically, during certain ophthalmic procedures, a gas such as SF₆ or a perfluorinated carbon, e.g., CF₄, C₂F₆, or C₃F₈, is mixed with air manually by a user (e.g., a technician) in preparation for administration to the patient. For example, depending on the application, commonly used gas mixtures include 30% SF₆, 20% C₂F₆, and 15% C₃F₈, each diluted in air. There is, however, no existing mechanism to accurately and efficiently verify one or more parameters, e.g., density, of the mixed gas prior to ophthalmic administration.

Many of the apparatus and methods described herein generally operate based on thermal conductivity. Heavy gases, such as SF₆ and perfluorinated fluorocarbons, are better heat conductors than air. Accordingly, measuring the thermal conductivity of a gas sample can help verify the presence of heavy gases. Thus, the thermal conductivity of a gas sample is an indicator of a parameter of the gas sample. Such parameters of the gas sample can include density and/or concentration.

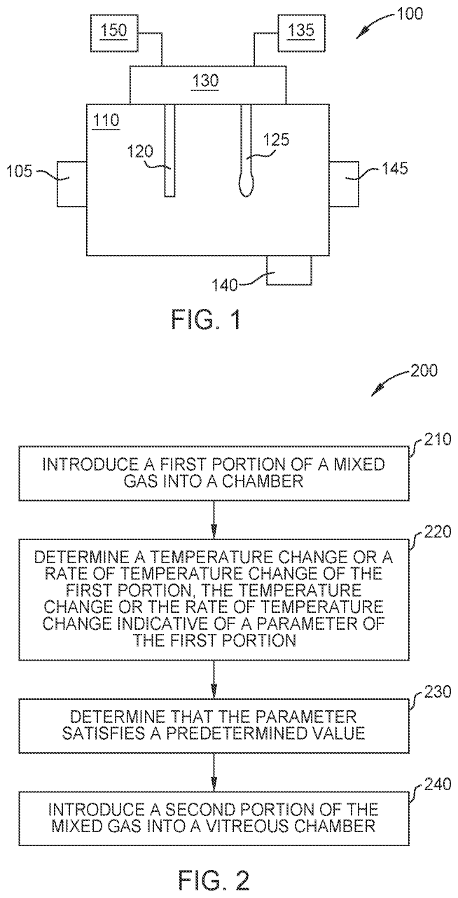

FIG. 1 is an example apparatus 100 for verifying a parameter, e.g., density and/or concentration, of a mixed gas according to at least one embodiment of the present disclosure. The apparatus 100 includes a sample port 105 where the mixed gas is introduced. The sample port 105 is coupled to chamber 110. Chamber 110 can be sized so that the gas sampling does not significantly reduce the amount of gas available for the ophthalmic procedure. The chamber 110 houses heater wire 120 (e.g., a resistance heating element) and temperature sensor 125 (e.g., thermometer, thermocouple, or resistance temperature detector (RTD)). The heater wire 120 is used to heat the sample and the temperature sensor 125 measures the temperature. The heater wire (or ribbon) can include any suitable material such as a metal, a noble metal, and/or a metal alloy, such as platinum, rhodium, nickel, chromium, tantalum, tungsten, or a combination thereof. The temperature sensor 125 can include any suitable material such as a metal, a noble metal, and/or a metal alloy, e.g., platinum, nickel, copper, or a combination thereof.

Controller 130 includes components such as hardware and circuitry to control the heater wire 120 and temperature sensor 125. Controller 130 is also configured to detect a parameter, directly or indirectly, of the mixed gas injected into the sample port 105. Controller 130 can include a processor. The processor can include memory to store instructions for various methods and operations described herein.

The apparatus 100 is powered by power supply 135, e.g., batteries, AC (alternating current) power supply, DC (direct current) power supply, and the like. Purge gas port 140 and purge vent 145 are coupled to chamber 110. The purge gas port 140 provides an exit for the gas sample after measurement. A filter can be placed at a position along the path from the sample port 105 to the purge gas port 140. Purge vent 145 is used to purge gas from the chamber 110. For example, the chamber 110 can contain a previous gas sample or air and so the chamber 110 can be flushed with, e.g., dry nitrogen, to a known initial condition. As an alternative to purge vent 145, a vacuum can be used to purge the chamber prior to introduction of the mixed gas. The apparatus 100 further includes an indicator 150. The indicator 150 provides a signal, e.g., a light, that provides an indication to the user that the mixed gas satisfies (or does not satisfy) a predetermined density or concentration. Controller 130 includes components, e.g., hardware and circuitry, to control the indicator 150.

By using apparatus 100, there are several ways that the heat conduction of the mixed gas can be measured. A direct measurement can be made by introducing a known heat source to the chamber 110 and measuring the heat rise in the chamber 110. The amount or rate of heat rise detected is an indication of a parameter of the mixed gas. For example, the heat can be provided by an electrically-heated element, e.g., heater wire 120, controlled electronically to produce heat energy at a known rate. The temperature is then determined, e.g., measured, by a temperature sensor, e.g., temperature sensor 125. The rate of temperature change and/or amount of temperature change is used to determine a parameter, e.g., a density and/or concentration, of the mixed gas.

FIG. 2 is a flowchart of an example method 200 of verifying a parameter, e.g., density and/or concentration, of a mixed gas according to at least one embodiment of the present disclosure. The method 200 can be used with apparatus 100, although apparatus 100 is only an example of an apparatus that may be used in conjunction with method 200. The method begins with a user preparing a volume of a mixed gas (e.g., SF₆ and air) in an amount sufficient for both the ophthalmic administration and the characterization. That is, a first portion of the volume of mixed gas is used to verify whether the mixed gas is satisfactory, and a second portion of the volume of mixed gas is used for administration into a patient's eye (e.g., the vitreous chamber of the patient's eye). The method includes introducing the mixed gas into a chamber at operation 210. As an example, the mixed gas is introduced into chamber 110 via sample port 105.

The method further includes determining a temperature change or rate of temperature change of the mixed gas at operation 220. Determining a temperature change or rate of temperature change can include heating the mixed gas to a heated mixed gas and measuring a temperature of the heated mixed gas. As an example, the heat can be provided by an electrically-heated element, e.g., heater wire 120, controlled electronically to produce heat energy at a known rate. The temperature is then determined, e.g., measured, by a temperature sensor, e.g., temperature sensor 125. The rate of temperature change and/or amount of temperature change is indicative of a parameter, e.g., density, of the mixed gas.

The method 200 further includes determining that the parameter (e.g., density and/or concentration) satisfies a predetermined value at operation 230. Here, the parameter can be compared to a predetermined value. Such an operation can be performed by the controller 130. For example, a readout system (e.g., as part of controller 130) connected to temperature sensor 125 can be used to measure the temperature signal. The readout system, which can include an temperature analyzer, can be integrated in a small dedicated device, it can be part of an integrated circuit, a sensor-on-chip, etc. The readout system can include analog-to-digital converters, memory modules and look-up tables, displays, etc. The processor of the controller 130 can then compare the measurement provided by the readout system with known tables of gases or calibration data and the density and/or concentration of mixed gas can be obtained. If the parameter satisfies the predetermined value, operation 240 can be performed. Operation 240 includes introducing the mixed gas into a patient's eye (e.g., the vitreous chamber of the patient's eye). If the parameter does not satisfy the predetermined value, then the user would prepare another mixed gas for method 200. An indication of whether a satisfactory mixed gas was prepared can be provided by indicator 150. As a non-limiting example, indicator 150 can illuminate as, e.g., a certain color based on whether the mixed gas is satisfactory. Alternatively, and as a non-limiting example, indicator 150 can provide a yes/no (+/−) message as to whether the mixed gas is satisfactory.

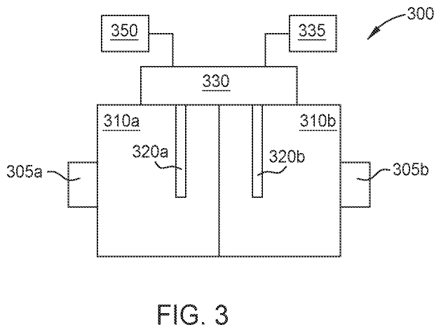

FIG. 3 is an example apparatus 300 for verifying a parameter, e.g., density and/or concentration, of a mixed gas according to at least one embodiment of the present disclosure. The apparatus 300 includes a sample chamber 310a and a reference chamber 310b.

The apparatus 300 includes a sample port 305a where the mixed gas is introduced. The sample port 305a is coupled to sample chamber 310a. Sample chamber 310a can be sized so that the gas sampling does not significantly reduce the amount of gas available for the ophthalmic procedure. The apparatus 300 includes a reference sample port 305b where a reference sample is introduced. Reference sample port 305b is coupled to reference chamber 310b. Sample chamber 310a houses a sample heater and sensor 320a. The sample heater and sensor 320a includes components to heat the sample and measure the temperature of the sample. The sample heater and sensor 320a can include a heater wire (e.g., a resistance heating element) and a temperature sensor, e.g., a thermometer, thermocouple, or RTD. Characteristics of the heater wire and temperature sensor are discussed above. Reference chamber 310b houses a reference heater and sensor 320b. The reference heater and sensor 320b includes components to heat the reference sample and measure the temperature of the reference sample. The reference heater and sensor 320b can include a heater wire (e.g., a resistance heating element) and a temperature sensor, e.g., a thermometer, thermocouple, or RTD. Characteristics of the heater wire and temperature sensor useful for reference heater and sensor 320b are discussed above in relation to FIG. 1.

Controller 330 includes components such as hardware and circuitry to control the sample heater and sensor 320a and the reference heater and sensor 320b. Controller 330 is also configured to detect a parameter, directly or indirectly, of the mixed gas injected into the sample port 305a and a parameter of the reference sample injected into the reference sample port 305b. Controller 330 can include a processor. The processor can include memory to store instructions for various methods and operations described herein. The apparatus 300 is powered by power supply 335, e.g., batteries, AC power supply, DC power supply, and the like. Each of the sample chamber 310a and the reference chamber 310b, independently, can be coupled to a purge gas port, a purge vent, and/or a vacuum. The apparatus 300 further includes an indicator 350. The indicator 350 provides a signal, e.g., a light, that provides an indication to the user that the mixed gas satisfies (or does not satisfy) a predetermined density or concentration. Controller 130 includes components such as hardware and circuitry to control the indicator 350.

By using apparatus 300, there are several ways that the heat conduction of the mixed gas can be measured. As an example, and during operation, parameters of the sample chamber 310a and the reference chamber 310b are, independently, set to expose the mixed gas and the reference sample to the same environmental conditions. The initial temperatures of the sample chamber 310a and the reference chamber 310b would be the same. Similarly, the heat loss from the sample chamber 310a and the reference chamber would be the same. The reference chamber 310b can contain a reference sample of a gas, e.g., dry nitrogen, and the sample chamber 310a can contain the mixed gas to be measured. Both the mixed gas and the reference sample are then subjected to the same heating and measurement method via sample heater and sensor 320a and the reference heater and sensor 320b, respectively. The difference in the rates (or amount) of heating between the mixed gas and the reference sample is then determined. Such difference in the rates can be used to determine a parameter, e.g., a density and/or concentration of the mixed gas. This differential measurement can eliminate the need for absolute accuracy in the measurement.

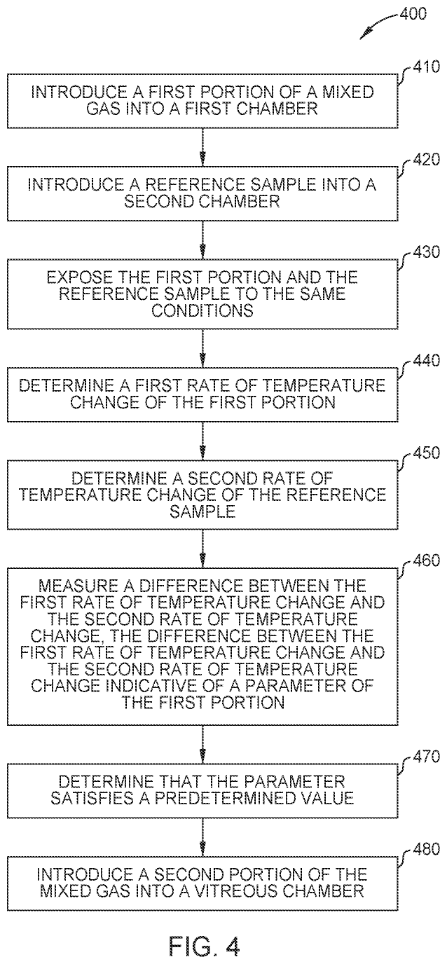

FIG. 4 is a flowchart of an example method 400 of verifying a parameter, e.g., density and/or concentration, of a mixed gas according to at least one embodiment of the present disclosure. The method 400 can be used with apparatus 300, although apparatus 300 is only an example of an apparatus that may be used in conjunction with method 400. The method begins with a user preparing a volume of a mixed gas (e.g., SF₆ and air) in an amount sufficient for both the ophthalmic administration and the characterization. That is, a first portion of the volume of mixed gas is used to verify whether the mixed gas is satisfactory, and a second portion of the volume of mixed gas is used for administration into a patient's eye (e.g., the vitreous chamber of the patient's eye).

The method 400 includes introducing a mixed gas into a sample chamber at operation 410. As an example, a mixed gas is introduced into sample chamber 310a via sample port 305a. Here, a volume of mixed gas (e.g., SF₆ and air) can be prepared in an amount sufficient for both the ophthalmic administration and the characterization. Method 400 further includes introducing a reference sample into a reference chamber at operation 420. Here a volume of reference sample (e.g., nitrogen gas) can be introduced into reference chamber 310b via reference sample port 305b. Alternatively, the reference gas can be introduced periodically to the reference chamber 310b. Periodic introduction of the reference gas can depend on the stability of the reference gas used and/or whether leakage (and rate thereof) of the reference gas occurs.

Method 400 further includes exposing the mixed gas and the reference sample to the same (or similar) environmental conditions at operation 430. Environmental conditions can include, but are not limited to, humidity, temperature, and pressure. For example, operation 430 can include setting parameters of the sample chamber 310a and the reference chamber 310b to predetermined values, such that the mixed gas and the reference sample are subject to the same or similar environmental conditions. By subjecting the reference sample and the mixed gas to the same or similar conditions, a correction factor for, e.g., local atmospheric pressure, local temperature, and local humidity can be made in order to null out the environment.

Method 400 further includes determining a temperature change or rate of temperature change of the mixed gas at operation 440. Determining a temperature change or rate of temperature change of the mixed gas can include heating the mixed gas to a heated mixed gas and measuring a temperature of the heated mixed gas. As an example, the heat can be provided by an electrically-heated element, e.g., sample heater and sensor 320a. The temperature is then determined, e.g., measured, by a temperature sensor, e.g., sample heater and sensor 320a. Method 400 further includes determining a temperature change or rate of temperature change of the reference sample at operation 450. Determining a temperature change or rate of temperature change of the reference sample can include heating the reference sample to a heated reference sample and measuring a temperature of the heated reference sample. As an example, the reference heater and sensor 320b includes components to heat the reference sample and measure the temperature of the reference sample.

Method 400 further includes measuring a difference between the rate of temperature change of the mixed gas (e.g., a first rate) and the rate of temperature change of the reference sample (e.g., a second rate) at operation 460. The difference between the rates of temperature change is indicative of a parameter of the mixed gas. Alternatively, or additionally, operation 460 can include measuring a difference between the temperature change of the mixed gas (e.g., a first amount) and the temperature change of the reference sample (e.g., a second amount). The difference between the amounts of temperature change is indicative of a parameter of the mixed gas. Such parameters include density and/or concentration. Determination of the density and concentration based on temperature is described above.

The method 400 further includes determining that the parameter (e.g., density and/or concentration) of the mixed gas satisfies a predetermined value at operation 470. Here, the parameter (e.g., density) can be compared to a predetermined value. Such an operation can be performed by the controller 330. For example, a readout system (e.g., as part of controller 330) connected to sample heater and sensor 320a and reference heater and sensor 320b can be used to measure the temperature signal. The readout system, which can include a temperature analyzer, can be integrated in a small dedicated device, it can be part of an integrated circuit, a sensor-on-chip, etc. The readout system can include analog-to-digital converters, memory modules and look-up tables, displays, etc. If the parameter satisfies the predetermined value, operation 480 can be performed. Operation 480 includes introducing a portion, e.g., a second portion, of the mixed gas into a patient's eye (e.g., the vitreous chamber of the patient's eye). If the parameter does not satisfy the predetermined value, then the user would prepare another mixed gas for method 400. An indication of whether a satisfactory mixed gas was prepared can be provided by indicator 350. As a non-limiting example, indicator 350 can illuminate as, e.g., a certain color based on whether the mixed gas is satisfactory. Alternatively, and as a non-limiting example, indicator 350 can provide a yes/no (+/−) message as to whether the mixed gas is satisfactory.

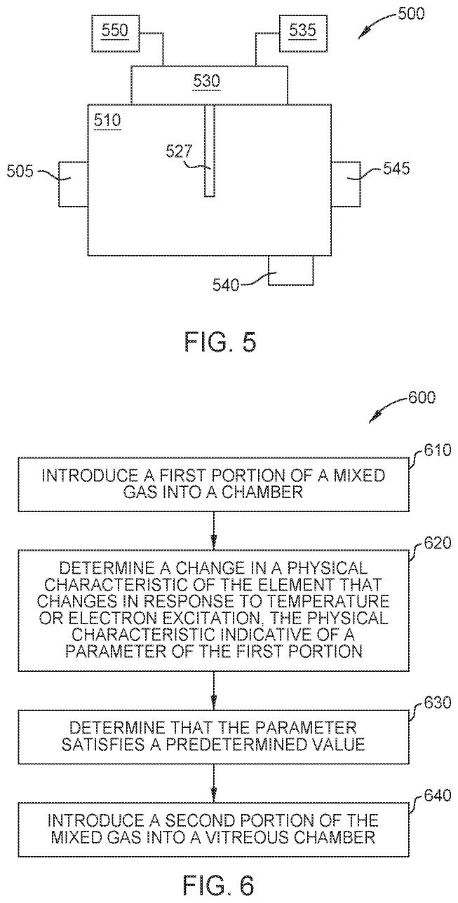

FIG. 5 is an example apparatus 500 for verifying a parameter, e.g., density and/or concentration, of a mixed gas according to at least one embodiment of the present disclosure. The apparatus 500 includes a sample port 505 where the mixed gas is introduced. The sample port 505 is coupled to chamber 510. Chamber 510 can be sized so that the gas sampling does not significantly reduce the amount of gas available for the ophthalmic procedure. The chamber 510 houses an element 527. In some embodiments, the element 527 has physical characteristics that change in response to temperature and/or electrical excitation. Such physical characteristics can include, but are not limited to, resistance, impedance, and oscillation.

In some embodiments, the element 527 includes an electrical element, e.g., such as a wire and/or a ribbon made of a material comprising a metal, a noble metal, and/or a metal alloy, such as platinum, copper, tungsten, platinum, rhodium, nickel, chromium, tantalum, tungsten, copper, or a combination thereof. The element 527 can provide both heating functionality and temperature measurement functionality. In some embodiments, element 527 includes an oscillator that changes in response to electronic excitation. The oscillator can be made of any suitable material, e.g., glass such as borosilicate glass, metals, and/or metal alloys, with oscillation capacity.

Controller 530 includes components such as hardware and circuitry to control the element 527. The processor can include memory to store instructions for various methods and operations described herein. Controller 530 is also configured to detect a parameter, directly or indirectly, of the mixed gas injected into the sample port 505 and/or configured to detect, directly or indirectly, a physical characteristic of the element 527. Controller 530 can include a processor. The apparatus 500 is powered by power supply 535, e.g., batteries, AC power supply, DC power supply, and the like. Purge gas port 540 and purge vent 545 are coupled to chamber 510. The purge gas port 540 provides an exit for the gas sample after measurement. A filter can be placed at a position along the path from the sample port 505 to the purge gas port 540. Purge vent 545 is used to purge gas from the chamber 510. For example, the chamber 510 can contain a previous gas sample or air and so the chamber 510 can be flushed with, e.g., dry nitrogen, to a known initial condition. As an alternative to purge vent 545, a vacuum can be used to purge the chamber prior to introduction of the mixed gas. The apparatus 500 further includes an indicator 550. The indicator 550 provides a signal, e.g., a light, that provides an indication to the user that the mixed gas satisfies (or does not satisfy) a predetermined density or concentration. Controller 530 includes components, e.g., hardware and circuitry, to control the indicator 550.

By using apparatus 500, there are several ways that the mixed gas can be verified, such as resistance change, impedance change, and/or oscillation change of element 527, as further described in relation to FIG. 6.

FIG. 6 is a flowchart of an example method 600 of verifying a parameter, e.g., density and/or concentration, of a mixed gas according to at least one embodiment of the present disclosure. The method 600 can be used with apparatus 500, although apparatus 500 is only an example of an apparatus that may be used in conjunction with method 600. The method begins with a user preparing a volume of a mixed gas (e.g., SF₆ and air) in an amount sufficient for both the ophthalmic administration and the characterization. That is, a first portion of the volume of mixed gas is used to verify whether the mixed gas is satisfactory, and a second portion of the volume of mixed gas is used for administration into a patient's eye (e.g., the vitreous chamber of the patient's eye). The method 600 includes introducing a mixed gas into a chamber at operation 610. As an example, a mixed gas is introduced into chamber 510 via sample port 505.

Method 600 further includes determining a change in a physical characteristic (e.g., resistance, impedance, oscillation) of an element (e.g., element 527, such as an electrical element and/or an oscillator) that changes in response to a stimulus, e.g., temperature or electron excitation, at operation 620. Determining the resistance, the impedance, and the oscillation are further described below. As also described below, the change in physical characteristic is indicative of a parameter, e.g., a density and/or concentration, of the mixed gas. Method 600 further includes determining that the parameter (e.g., density and/or concentration) satisfies a predetermined value at operation 630. Here, the parameter (e.g., density) of the mixed gas can be compared to a predetermined value. Such an operation can be performed by the controller 530. If the parameter satisfies the predetermined value, operation 640 can be performed. Operation 640 includes introducing a portion of the mixed gas into a patient's eye (e.g., the vitreous chamber of the patient's eye). If the parameter does not satisfy the predetermined value, then the user would prepare another mixed gas for method 600. An indication of whether a satisfactory mixed gas was prepared can be provided by indicator 550. As a non-limiting example, indicator 550 can illuminate as, e.g., a certain color based on whether the mixed gas is satisfactory. Alternatively, and as a non-limiting example, indicator 550 can provide a yes/no (+/−) message as to whether the mixed gas is satisfactory.

As stated above, various physical characteristics of an element (e.g., element 527) can be determined such as resistance, impedance, and oscillation.

Resistance can be measured, and the resistance can be related to the density and/or concentration of the mixed gas. The resistance of a metal wire changes with temperature, so the wire can be used as the thermometer. As the wire is heated while exposed to a gas, the wire loses heat through conduction which is proportional to the density of the gas.

In some embodiments, the element 527 is an electrical element comprising a material having a known electrical resistance-to-temperature relationship such as a metal, a metal alloy, or a combination thereof, such as platinum, copper, tungsten, platinum, rhodium, nickel, chromium, tantalum, tungsten, copper, or a combination thereof. As the element 527 is electrically heated, its resistance change can be determined. The change in resistance is proportional to how much heat is being applied and how much heat is removed by the thermal conduction of the gas surrounding the element 527. Accordingly, determining the change of resistance (or rate of change of resistance) with a known heating rate is used to determine a parameter, e.g., a density and/or concentration, of the mixed gas.

Generally for resistance measurements, the element 527 (e.g., a wire) can be exposed to gas. An electrical signal is passed through element 527. Here, the element 527 can be heated by applying a constant voltage over the element, and the element's 527 resistance response is measured, e.g., for a period of time. For example, a readout system (e.g., as part of controller 530) connected to electrodes of the element 527 can be used to measure the resistance response signal. The readout system, which can include an resistance analyzer, can be integrated in a small dedicated device, it can be part of an integrated circuit, a sensor-on-chip, etc. The readout system can include analog-to-digital converters, memory modules and look-up tables, displays, etc. Both AC and DC circuits can be used. The processor of the controller 530 can then compare the measurement with known tables of gases or calibration data and the density and/or concentration of mixed gas can be obtained.

The element's 527 electrical resistance increases as the element's temperature increases, which can vary with the electrical current flowing through the circuit according to Ohm's law (V=IR), where V is the voltage, I is the current, and R is the resistance. When a gas flows past the element, the element cools, decreasing its resistance, which in turn allows more current to flow through the circuit, since the supply voltage is a constant. As more current flows, the element's temperature increases until the resistance reaches equilibrium again. The current increase or decrease is proportional to the mass of gas flowing past the wire. An integrated electronic circuit converts the proportional measurement into a calibrated signal which is sent to the controller 530. The change in resistance can then be determined since the current is known and the resulting voltage reveals the resistance.

Impedance can also be measured, and the impedance can be related to the density and/or concentration of the mixed gas. Here, the element 527 is an electrical element comprising a material having a known electrical impedance-to-temperature relationship such as a metal, a metal alloy, or a combination thereof, such as platinum, copper, tungsten, platinum, rhodium, nickel, chromium, tantalum, tungsten, copper, or a combination thereof. As the element is heated, its impedance change can be determined. The change in impedance is proportional to how much heat is being applied and how much heat is removed by the thermal conduction of the gas surrounding the element. Determining the change of impedance (or the rate of change of impedance) with a known heating rate is used to determine a parameter, e.g., a density and/or concentration, of the mixed gas.

For impedance measurements, the element 527 can be exposed to the gas. An electrical signal is passed through the element 527. Here, element 527 is then polarized with a variable electrical signal at a predetermined frequency or range of frequencies, and the impedance response is measured, e.g., for a period of time. For example, a readout system (e.g., as part of controller 530) connected to electrodes of the element 527 can be used to measure the impedimetric response signal. The readout system, which can include an impedance analyzer, can be integrated in a small dedicated device, it can be part of an integrated circuit, a sensor-on-chip, etc. The processor of controller 530 can then compare the measurement with known tables of gases or calibration data and the density and/or concentration of mixed gas can be obtained. The measurement can be repeated for different frequencies.

The element's 527 response to the electrical signal can take into account the combination of a resistive component (R) and a capacitive component (C) of impedance. For example, the resistive component can be obtained after or for a period of time, and the capacitive component can also be obtained after a period of time, or the capacitive component can be recorded for a period of time. The impedance response can be frequency-dependent, and the contributions of the resistive component R and the capacitive component C to the change in impedance (Z) can vary for particular element 527/gas pairs. The components C and R can be obtained by measuring the impedance on the element 527. Additionally, the readout system connected to electrodes of the element 527 can be used to separately measure the capacitive and resistive components. For example, C, R, or both can be measured for a range of frequencies, or the variation of the impedance (e.g., the change of C, or R, or both) with time can be obtained for a frequency or a range of frequencies. The readout system can include analog-to-digital converters, memory modules and look-up tables, displays, etc. Both AC and DC circuits can be used.

Oscillation characteristics of an element 527 can also be measured when an oscillator is used for element 527. Use of an oscillator allows determination of the density and/or concentration of a mixed gas based on an electronic measurement of the frequency of oscillation, from which the density value can be calculated. Various types of oscillators can be used such as a Y-oscillator, an X-oscillator, and a W-oscillator. Y-oscillators, which are typical U-tubes, move up and down and typically use a counter mass to dampen or eliminate harmful vibrations. X-oscillators are U-tubes with a fixed bend, and the moving parts move towards each other in opposite directions. W-oscillators are characterized by three bends, the first and last bend oscillate towards each other in opposite directions. The oscillator can be made of any suitable material, e.g., a glass tube such as borosilicate glass, metals, and/or metal alloys, with oscillation capacity.

Generally, for oscillation measurements, the element 527 (e.g., an oscillator) can be exposed to the mixed gas. As an example, a mixed gas is filled into a hollow oscillator such as a U-tube. The oscillator is then electronically excited into an un-damped oscillation and the period of oscillation of the oscillator can be measured. For example, a readout system (e.g., as part of controller 530) connected to element 527 can be used to measure the oscillation response signal. The readout system, which can include an oscillation analyzer, can be integrated in a small dedicated device, it can be part of an integrated circuit, a sensor-on-chip, etc. The readout system can include analog-to-digital converters, memory modules and look-up tables, displays, etc. The processor of the controller 530 can then compare the measurement with known tables of gases or calibration data and the density and/or concentration of mixed gas can be obtained.

Piezoelements (e.g., crystal or ceramic material), or a system of magnets and coils, can be used to excite the U-tube and optical pickups can measure the period of oscillation. The optical pickups can detect a light beam that is interrupted by a coating on the oscillating U-tube and the pickups record the oscillation period. Piezoelements can also be used to represent the period of oscillation when, e.g., the usable effect of the element is inverted. While the excitation of the oscillator is enabled by applying electrical voltage to the Piezoelement, detection of the oscillation can be performed by the following: a second piezoelement is then pressurized by the moving sensor unit periodically and generates electric voltage that represents the period of oscillation very accurately. Magnets can be used to measure the period of oscillation as well. Whenever a magnet passes the coil, a current is induced which can be evaluated.

Pre-calibration operations can be performed for the measurements described herein. For example, the measurement of resistance changes and impedance changes with temperature and in isolation of gasses, for temperature compensation, or measurement of known gaseous analytes at known concentrations and/or densities can be performed. As another example, the measurement of oscillation changes with electron excitation in isolation of gases, for electron excitation compensation, or measurement of known gaseous analytes at known concentrations and/or densities can be performed. As another example, the measurement of temperature changes and rates of temperature changes in isolation of gasses or with measurement of known gaseous analytes at known concentrations and/or densities can be performed.

The apparatus described herein (e.g., apparatus 100, apparatus 300, and apparatus 500) can be used with other tools, such as Constellation™ commercially available from Alcon Laboratories, Inc., Fort Worth, Texas. In some embodiments, the methods described herein (e.g., method 200, method 400, and method 600) can be a portion of a method for ophthalmic-based therapies, such as retinal detachment repair. Accordingly, after verifying a parameter, e.g., density and/or concentration, a portion of a mixed gas (e.g., determining that the parameter satisfies a predetermined value), and another portion of a mixed gas can be introduced into a patient's eye (e.g., the vitreous chamber of the patient's eye).

According to at least one embodiment, one or more operations of the methods described above can be included as instructions in a tangible, non-transitory computer-readable medium for execution by a control unit (e.g., controller 130, controller 330, and controller 530 which can each include a processor) or any other processing system. The computer-readable medium can include any suitable memory for storing instructions, such as read-only memory (ROM), random access memory (RAM), flash memory, an electrically erasable programmable ROM (EEPROM), a compact disc ROM (CD-ROM), a floppy disk, punched cards, magnetic tape, and the like.

### Embodiments Listing

The present disclosure provides, among others, the following aspects, each of which may be considered as optionally including any alternate aspects.
Clause 1. A method, comprising: preparing a volume of mixed gas, the volume of mixed gas comprising a first portion and a second portion; introducing the first portion into a chamber; determining a temperature change or a rate of temperature change of the first portion, the temperature change or the rate of temperature change indicative of a parameter of the first portion; determining that the parameter satisfies a predetermined value; and introducing the second portion into a patient's eye (e.g., the vitreous chamber of the patient's eye).
Clause 2. The method of Clause 1, wherein determining a temperature change or a rate of temperature change comprises heating the first portion to a heated first portion and measuring a temperature of the heated first portion.
Clause 3. The method of Clause 1 or Clause 2, wherein the parameter comprises a density, a concentration, or a combination thereof.
Clause 4. The method of any one of Clauses 1-3, wherein the volume of mixed gas comprises SF₆, CF₄, C₂F₆, C₃F₈, or a combination thereof.
Clause 5. A method, comprising: preparing a volume of mixed gas, the volume of mixed gas comprising a first portion and a second portion; introducing the first portion into a first chamber; introducing a reference sample into a second chamber; exposing the first portion and the reference sample to the same conditions; determining a first rate of temperature change of the first portion; determining a second rate of temperature change of the reference sample; measuring a difference between the first rate of temperature change and the second rate of temperature change, the difference between the first rate of temperature change and the second rate of temperature change indicative of a parameter of the first portion; determining that the parameter satisfies a predetermined value; and introducing the second portion into a patient's eye (e.g., the vitreous chamber of the patient's eye).
Clause 6. The method of Clause 5, wherein: determining a first rate of temperature change comprises heating the first portion to a heated first portion and measuring a temperature of the heated first portion; determining a second rate of temperature change comprises heating the reference sample to a heated reference sample and measuring a temperature of the heated reference sample; or a combination thereof.
Clause 7. The method of Clause 5 or Clause 6, wherein the parameter comprises a density, a concentration, or a combination thereof.
Clause 8. The method of any one of Clauses 5-7, wherein the volume of mixed gas comprises SF₆, CF₄, C₂F₆, C₃F₈, or a combination thereof.
Clause 9. A method, comprising: preparing a volume of mixed gas, the volume of mixed gas comprising a first portion and a second portion; introducing the first portion into a chamber, the chamber housing an element; determining a change in a physical characteristic of the element that changes in response to temperature or electron excitation, the physical characteristic indicative of a parameter of the first portion; determining that the parameter satisfies a predetermined value; and introducing the second portion into a patient's eye (e.g., the vitreous chamber of the patient's eye).
Clause 10. The method of Clause 9, wherein the element is an electrical element configured to heat a gas, measure a temperature of a gas, or a combination thereof.
Clause 11. The method of Clause 9 or Clause 10, wherein the physical characteristic that changes in response to temperature includes resistance, impedance, or a combination thereof.
Clause 12. The method of any one of Clauses 9-11, wherein the physical characteristic is resistance, and determining a change in resistance comprises: heating the electrical element by applying a constant voltage; and measuring a response in resistance.
Clause 13. The method of any one of Clauses 9-12, wherein the physical characteristic is impedance, and determining a change in impedance comprises: passing an electrical signal through the electrical element; and measuring a response in impedance.
Clause 14. The method of any one of Clauses 9-13, wherein: the element is an oscillator, and the physical characteristic that changes in response to electron excitation includes oscillation.
Clause 15. The method of Clause 14, wherein determining a change in oscillation comprises: electronically exciting an oscillator; and measuring a period of oscillation.
Clause 16. The method of any one of Clauses 9-15, wherein the parameter comprises a density, a concentration, or a combination thereof.
Clause 17. The method of any one of Clauses 9-16, wherein the volume of mixed gas comprises SF₆, CF₄, C₂F₆, C₃F₈, or a combination thereof.

All documents described herein are incorporated by reference herein, including any priority documents and/or testing procedures to the extent they are not inconsistent with this text. As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby. Likewise, the term “comprising” is considered synonymous with the term “including.” Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

The term “coupled” is used herein to refer to elements that are either directly connected or connected through one or more intervening elements. For example, a gas sample port (e.g., where a gas sample is introduced) can be directly connected to the chamber, or it can be connected to the chamber via intervening elements.

What is claimed is:

1. A method, comprising:
preparing a volume of mixed gas, the volume of mixed gas comprising a first portion and a second portion;
introducing the first portion into a chamber, the chamber housing an element;
determining a change in a physical characteristic of the element that changes in response to temperature or electron excitation, the physical characteristic indicative of a parameter of the first portion;
determining that the parameter satisfies a predetermined value; and
introducing the second portion into a patient's eye.

2. The method of claim 1, wherein the element is an electrical element configured to heat a gas, measure a temperature of a gas, or a combination thereof.

3. The method of claim 2, wherein the physical characteristic that changes in response to temperature includes resistance, impedance, or a combination thereof.

4. The method of claim 3, wherein the physical characteristic is resistance, and determining a change in resistance comprises:
heating the electrical element by applying a constant voltage; and
measuring a response in resistance.

5. The method of claim 3, wherein the physical characteristic is impedance, and determining a change in impedance comprises:
passing an electrical signal through the electrical element; and
measuring a response in impedance.

6. The method of claim 1, wherein:
the element is an oscillator, and
the physical characteristic that changes in response to electron excitation includes oscillation.

7. The method of claim 6, wherein determining a change in oscillation comprises:
electronically exciting an oscillator; and
measuring a period of oscillation.
